(19) United States
(12) Patent Application Publication
(10) Pub. No.: US 2025/0099706 A1
(43) Pub. Date: Mar. 27, 2025

(54) CPAP APPARATUS

(71) Applicant: Murata Manufacturing Co., Ltd., Kyoto (JP)

(72) Inventor: Atsushi HATTORI, Kyoto (JP)

(21) Appl. No.: 18/976,538

(22) Filed: Dec. 11, 2024

Related U.S. Application Data
(63) Continuation of application No. PCT/JP2023/042702, filed on Nov. 29, 2023.

(30) Foreign Application Priority Data
Nov. 30, 2022 (JP) 2022-191516

(51) Int. Cl. A61M 16/10

(52) U.S. Cl. CPC A61M 16/1075

(57) ABSTRACT

A receiving unit of a CPAP apparatus includes a base member, a receiving case, a heating unit, and an elastic member. The receiving case is disposed on an upper side of the base member and includes an opening formed in a lower wall. The heating unit is disposed between the base member and the lower wall of the receiving case. The elastic member is disposed on the base member and holds the heating unit. The heating unit includes a heating element and a thermal conductor. The thermal conductor is disposed on an upper side of the heating element, has a shape wider than a shape of the heating element when the heating element is viewed from the receiving unit in plan view, and includes a peripheral portion that does not overlap the heating element.

### CROSS REFERENCE TO RELATED APPLICATION

This is a continuation of International Application No. PCT/JP2023/042702 filed on Nov. 29, 2023 which claims priority from Japanese Patent Application No. 2022-191516 filed on Nov. 30, 2022. The contents of these applications are incorporated herein by reference in their entireties.

### BACKGROUND OF THE DISCLOSURE

### Field of the Disclosure

The present disclosure relates to a CPAP apparatus including a main body receiving a humidification chamber.

### Description of the Related Art

Patent Document 1 discloses a CPAP system including a housing, a water reservoir, and a heating assembly. The heating assembly is mounted on a lower wall of the housing. The water reservoir is detachable from the housing. The water reservoir is disposed on an upper surface of the heating assembly in a state in which the water reservoir is mounted on the housing.

In the CPAP system of Patent Document 1, when the water reservoir is mounted on the housing, a gap for discharging water within the housing is formed, and undesired water within the housing is discharged to the outside of the housing.
Patent Document 1: Japanese Unexamined Patent Application Publication (Translation of PCT Application) No. 2022-529963

### BRIEF SUMMARY OF THE DISCLOSURE

However, in the CPAP system of Patent Document 1, it is difficult to discharge water within the housing unless the water reservoir is mounted.

Accordingly, it is a possible benefit of the present disclosure to provide a structure configured to discharge undesired water within a housing, irrespective of a state of a CPAP apparatus.

A CPAP apparatus according to an embodiment of the present disclosure includes a housing, a fan mechanism, a humidification chamber, and a receiving unit. The fan mechanism is provided in the housing and configured to generate a gas flow to be supplied to a user. The humidification chamber is detachably provided in the housing and configured to store water for humidifying the gas flow. The receiving unit is provided in the housing and receives the humidification chamber.

The receiving unit includes a base member formed by a portion of the housing or fixed to the housing, a receiving case, a heating unit, and an elastic member. The receiving unit is disposed on an upper side of the base member and includes an opening formed in a lower wall. The heating unit is disposed between the base member and the lower wall of the receiving case. The elastic member is disposed on the base member and holds the heating unit. The heating unit includes a heating element and a thermal conductor. The thermal conductor is disposed on an upper side of the heating element. The thermal conductor has a shape wider than a shape of the heating element when the heating element is viewed from the receiving unit in plan view. The thermal conductor includes a peripheral portion that does not overlap the heating element. The lower wall of the receiving case includes a plurality of first protruding portions formed on an outer peripheral portion of the thermal conductor in plan view and arranged at intervals in a peripheral direction of the outer peripheral portion. The receiving case and the thermal conductor are in contact with each other with the first protruding portions therebetween. Alternatively, the thermal conductor includes a plurality of second protruding portions formed on a portion overlapping an outer peripheral portion of the thermal conductor in plan view and arranged at intervals in a peripheral direction of the outer peripheral portion. The receiving case and the thermal conductor are in contact with each other with the second protruding portions therebetween.

In these configurations, there are gaps between the thermal conductor and the lower wall of the receiving case, except for the portions including the first protruding portions or the second protruding portions. These gaps are formed irrespective of the presence or absence of the humidification chamber in the receiving case. Accordingly, water on the upper surface of the lower wall of the receiving case is discharged through the gaps between the thermal conductor and the lower wall of the receiving case.

According to the present disclosure, it is possible to discharge undesired water within the housing, irrespective of a state of the CPAP apparatus.

### BRIEF DESCRIPTION OF THE SEVERAL VIEWS OF THE DRAWINGS

FIG. 1 is an external perspective view of a CPAP apparatus according to an embodiment of the present disclosure.

FIG. 2 is an external perspective view of the CPAP apparatus according to the embodiment of the present disclosure.

FIG. 3 is an external perspective view of a base unit according to the embodiment of the present disclosure.

FIG. 4 is a side sectional view illustrating a configuration of a portion corresponding to a receiving unit in the CPAP apparatus according to the embodiment of the present disclosure.

FIG. 5 is a side sectional view illustrating the configuration of the portion corresponding to the receiving unit in a box body according to the embodiment of the present disclosure.

FIG. 6 is a plan view of the box body according to the embodiment of the present disclosure.

FIG. 7A is a plan view of the box body, including a base member, according to the embodiment of the present disclosure. FIG. 7B is a side sectional view of this box body.

FIG. 8A is a plan view of a receiving case according to the embodiment of the present disclosure. FIG. 8B is a side sectional view of the receiving case.

FIG. 9A is a plan view of a thermal conductor according to the embodiment of the present disclosure. FIG. 9B is a side sectional view of the thermal conductor.

FIG. 10A is a plan view of an elastic member according to the embodiment of the present disclosure. FIG. 10B is a first side sectional view of the elastic member.

FIG. 10C is a second side view of the elastic member.

FIG. 11A and FIG. 11B are each an enlarged view of a side cross section for explaining a water discharge mechanism of the CPAP apparatus according to the embodiment of the present disclosure.

FIG. 12A and FIG. 12B are each an enlarged view of a side cross section illustrating a variation of protruding portions of the CPAP apparatus according to the embodiment of the present disclosure.

FIG. 13 is an enlarged view of a side cross section illustrating a variation of the protruding portions of the CPAP apparatus according to the embodiment of the present disclosure.

### DETAILED DESCRIPTION OF THE DISCLOSURE

A CPAP apparatus according to an embodiment of the present disclosure will be described with reference to the drawings.

### (Overview of CPAP Apparatus 10)

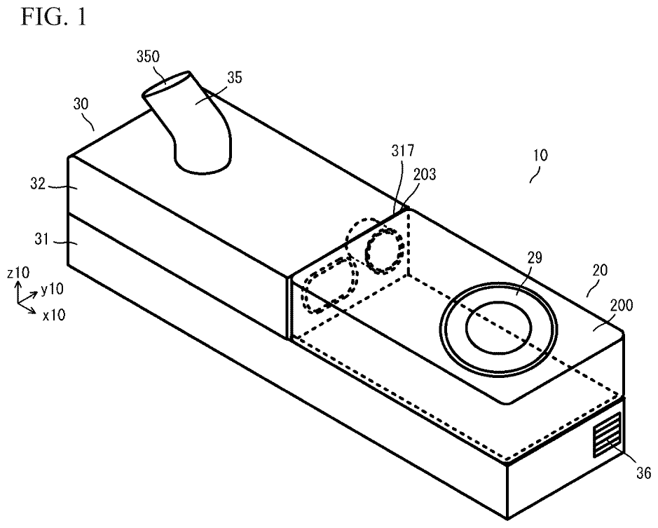

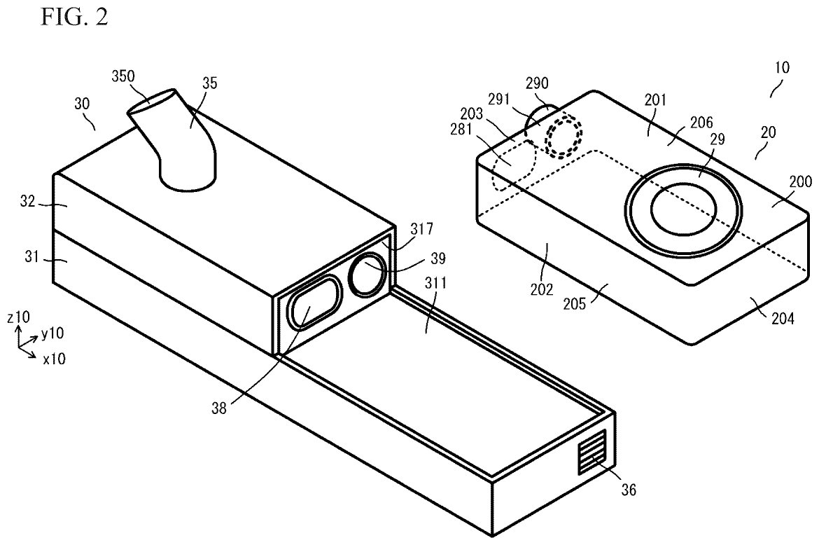

FIGS. 1 and 2 are each an external perspective view of the CPAP apparatus according to the embodiment of the present disclosure. FIG. 1 illustrates a state in which a main unit is mounted on a base unit, and FIG. 2 illustrates a state in which the main unit is detached from the base unit.

As illustrated in FIG. 1 and FIG. 2, a CPAP apparatus 10 includes a main unit 20 and a base unit 30. An outer shape of the main unit 20 is a rectangular parallelepiped. The main unit 20 has a height shorter than a length in a longitudinal direction and a length in a lateral direction. Note that “rectangular parallelepiped” in the present embodiment is not limited to a perfect rectangular parallelepiped and includes those whose vertices and edges are subjected to various types of chamfering.

The base unit 30 has, in plan view, a rectangular shape having a longitudinal direction and a lateral direction. The base unit 30 includes a tall portion and a short portion. The tall portion is taller than the short portion. That is, the base unit 30 has an outer shape in which a tall portion having a rectangular parallelepiped shape and a short portion having a rectangular parallelepiped shape are connected in the longitudinal direction of the base unit 30.

The main unit 20 can be mounted on an upper surface of the short portion of the base unit 30. In a state in which the main unit 20 is mounted on the base unit 30, the outer shape of the CPAP apparatus 10 is a rectangular parallelepiped.

In a state in which the main unit 20 is mounted on the base unit 30 (a mounted state), a gas flow passage of the main unit 20 and a gas flow passage of the base unit 30 communicate. Moreover, a first electric circuit of the main unit 20 and a second electric circuit of the base unit 30 are electrically connected. With this, the main unit 20 and the base unit 30 achieve a function of the CPAP apparatus 10 in cooperation with each other. In a state in which the main unit 20 is detached from the base unit 30, the main unit 20 operates alone to achieve the function of the CPAP apparatus 10. A box body, having a substantially rectangular parallelepiped shape, that is constituted in a state in which the main unit 20 is mounted on the base unit 30 corresponds to a form of a housing in the present disclosure.

The main unit 20 includes a fan (not shown) installed in the main unit 20. The CPAP apparatus 10 generates the gas flow to be supplied to the user by driving the fan.

### (Base Unit 30)

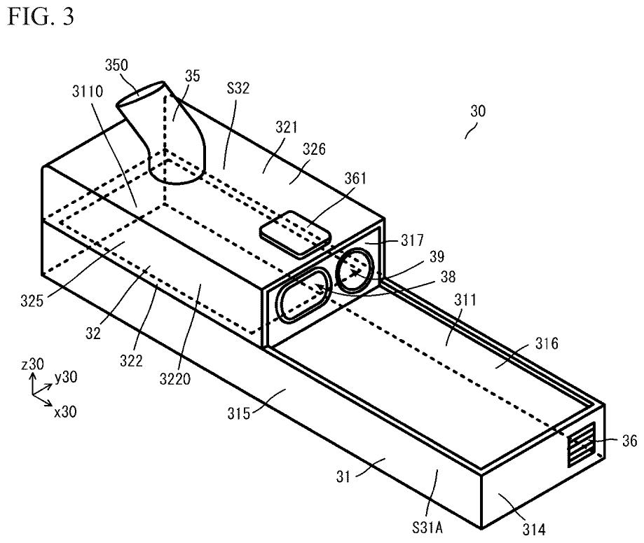

FIG. 3 is an external perspective view of a base unit according to the embodiment of the present disclosure. The base unit 30 corresponds to at least a portion of the housing in the present disclosure.

As illustrated in FIG. 3, the base unit 30 includes a box body 31 and a lid 32. Schematically speaking, a portion of an upper surface side of the box body 31 is opened. The lid 32 is pivotably connected to the box body 31. The base unit 30 is configured to be switched between the state in which the lid 32 covers the opening of the box body 31 and the state in which the lid 32 does not cover the opening of the box body 31. The state in which the lid 32 covers the opening of the box body 31 is used, for example, during use as the CPAP apparatus 10. The state in which the lid does not cover the opening of the box body 31 is used, for example, when a humidification chamber 40 received in the base unit 30 is supplied with a liquid.

### (Box Body 31)

The box body 31 includes an upper wall 311, a lower wall 312, a side wall 313, a side wall 314, a side wall 315, a side wall 316, and a side wall 317.

The upper wall 311 and the lower wall 312 have different dimensions in the longitudinal direction (the x30-axis direction), and have a substantially identical dimension in the lateral direction (the y30-axis direction). A portion of the lower wall 312 in the longitudinal direction faces the upper wall 311.

The side wall 313 and the side wall 314 have a substantially identical outer shape. The side wall 313 and the side wall 314 face each other. The side wall 313 and the side wall 314 are side walls which are parallel to the lateral direction of the base unit 30.

The side wall 315 and the side wall 316 have a substantially identical outer shape. The side wall 315 and the side wall 316 face each other. The side wall 315 and the side wall 316 are side walls which are parallel to the longitudinal direction of the base unit 30.

The side wall 313, the side wall 314, the side wall 315, and the side wall 316 are connected to four sides of the lower wall 312. The side wall 314, a portion of the side wall 315 in the longitudinal direction, and a portion of the side wall 316 in the longitudinal direction are connected to three sides of the upper wall 311. Here, the upper wall 311 and the side wall 313 are apart from each other in the longitudinal direction of the box body 31. Accordingly, the box body 31 includes the opening that is located on the upper surface side on which the upper wall 311 is disposed and that is located between the side wall 313 and an end portion of the upper wall 311 on the opposite side from the end portion connected to the side wall 314 in plan view.

The side wall 317 is disposed parallel to the side wall 313 and the side wall 314. The side wall 317 is connected to the end portion of the upper wall 311 on the opposite side from the end portion connected to the side wall 314.

Thus, the box body 31 has a space S31A defined by the upper wall 311, the lower wall 312, the side wall 314, the side wall 315, the side wall 316, and the side wall 317. At the same time, the box body 31 has a space S31B defined by the upper wall 311, the lower wall 312, the side wall 313, the side wall 315, the side wall 316, and the side wall 317. The space S31B is provided with an opening, on the upper surface side on which the upper wall 311 is disposed, between the side wall 317 and the side wall 313 in plan view. This portion forming the space S31B attains the receiving unit for the humidification chamber 40.

The side wall 314 has an inlet port 36. The inlet port 36 extends through the side wall 314 in a thickness direction.

The side wall 317 has an exhaust port 38 and an inlet port 39 which are located on an upper side of a portion connected to the upper wall 311. The exhaust port 38 extends through the inside of the side wall 317. The exhaust port 38 is connected with the space S31A. The inlet port 39 extends through the side wall 317 in the thickness direction. The exhaust port 38 and the inlet port 39 are arranged adjacent to each other in the widthwise direction of the side wall 317 (the y30-axis direction). An arrangement interval between the exhaust port 38 and the inlet port 39 is identical to an arrangement interval between an inlet port 281 and an exhaust port 291 of the main unit 20.

The side wall 317 includes a receiving member 3170 for a latch mechanism. The receiving member 3170 is positioned near an upper end of the side wall 317. The receiving member 3170 is formed on a wall surface of the side wall 317 on the opposite side from the upper wall 311 side.

### (Lid 32)

The lid 32 includes an upper wall 321, a side wall 323, a side wall 325, and a side wall 326. An outer shape of the upper wall 321 is substantially identical to a shape of the opening of the box body 31. An outer shape of the side wall 323 is substantially identical to an outer shape of the side wall 317 of the box body 31. The side wall 325 and the side wall 326 have a substantially identical outer shape. The side wall 325 and the side wall 326 face each other. The side wall 325 and the side wall 326 are side walls which are parallel to the longitudinal direction of the base unit 30.

The side wall 323, the side wall 325, and the side wall 326 are connected to three sides of the upper wall 321. With this, the lid 32 has a space S32 defined by the upper wall 321, the side wall 323, the side wall 325, and the side wall 326. Here, there is no wall facing the upper wall 311. Accordingly, the lid 32 has an opening on the side of the upper wall 321 on which the side wall 323, the side wall 325, and the side wall 326 are connected. In addition, there is no wall facing the side wall 323. Accordingly, the lid 32 has an opening on the side of the side wall 323 on which the upper wall 321, the side wall 325, and the side wall 326 are connected.

A gas blowing pipe 35 is formed on the upper wall 321. The gas blowing pipe 35 has a shape protruding outward from the upper wall 321. With the main unit 20 being mounted on the base unit 30, an opening 350 of the gas blowing pipe 35 serves as a final gas blowing port through which the desired gas (blow) is supplied to a user. Furthermore, an operator 361 for the latch mechanism is disposed on the upper wall 321. A hook 360 is connected to the operator 361. The hook 360 protrudes to the space S32 side relative to the upper wall 321.

The lid 32 includes a mechanism by which the lid 32 is pivoted with respect to the box body 31. With this, the CPAP apparatus 10 is configured to be switched between the state in which the lid 32 covers the opening of the box body 31 (the upper side of the receiving unit) and the state in which the lid 32 does not cover the opening of the box body 31 (the upper side of the receiving unit).

### (Receiving Unit)

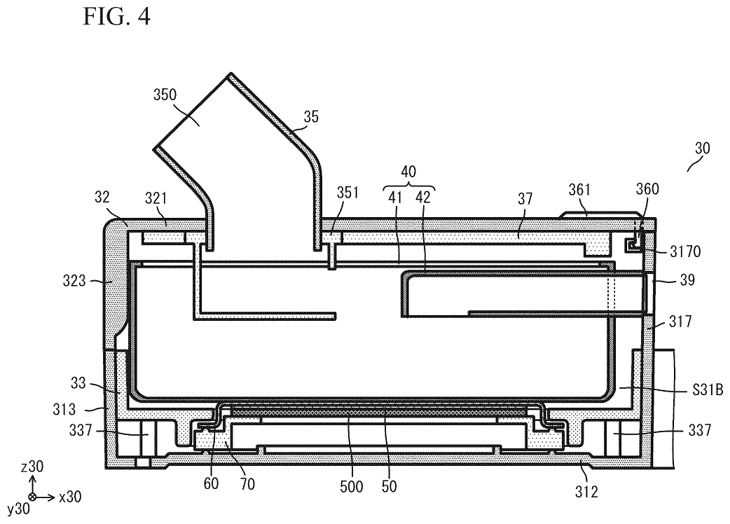

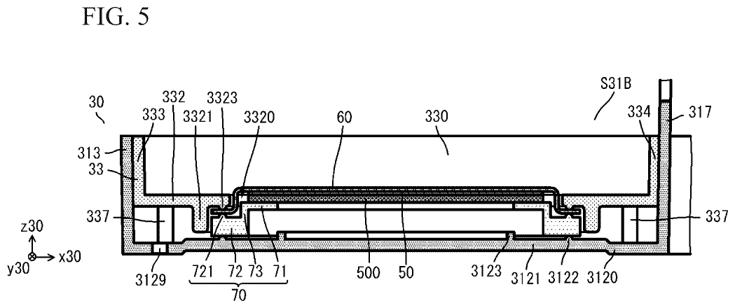

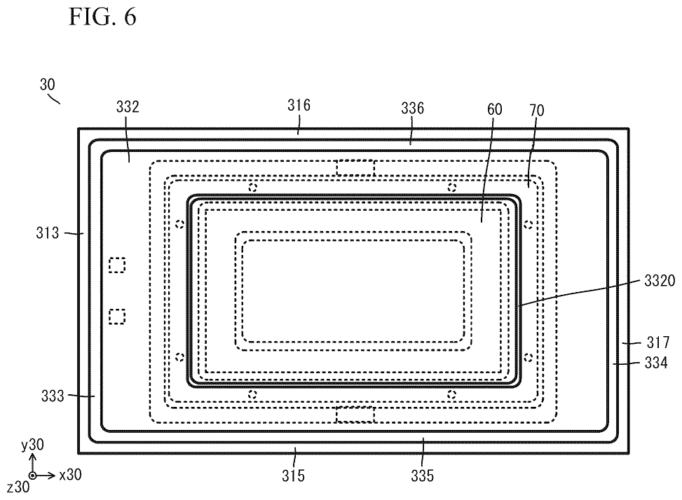

FIG. 4 is a side sectional view illustrating a configuration of a portion corresponding to the receiving unit in the CPAP apparatus according to the embodiment of the present disclosure. FIG. 5 is a side sectional view illustrating the configuration of the portion corresponding to the receiving unit in the box body according to the embodiment of the present disclosure. FIG. 4 and FIG. 5 are not sectional views obtained by cutting the CPAP apparatus and the box body in one plane, but are sectional views illustrated for readily understanding the configuration. FIG. 6 is a plan view of the box body according to the embodiment of the present disclosure.

As illustrated in FIG. 4, FIG. 5, and FIG. 6, the base unit 30 of the CPAP apparatus 10 is provided with a receiving case 33, the humidification chamber 40, a heating element 50, a circuit board 500, a thermal conductor 60, and an elastic member 70. The heating element 50 and the thermal conductor 60 constitute a heating unit in the present disclosure.

The thermal conductor 60, the heating element 50, the circuit board 500, and the elastic member 70 are disposed between the receiving case 33 and the lower wall 312 (the base member). The elastic member 70 is disposed on the lower wall 312 (the base member). The elastic member 70 is in contact with the lower wall 312. The circuit board 500 is disposed on the upper surface side of the elastic member 70. The heating element 50 is disposed on the upper surface side of the circuit board 500. The heating element 50 is electrically connected to the circuit board 500. The thermal conductor 60 is disposed on the upper surface side of the heating element 50. The thermal conductor 60 is in surface contact with the heating element 50. The receiving case 33 covers a multilayer body of the elastic member 70, the circuit board 500, the heating element 50, and the thermal conductor 60 from the upper side. The receiving case 33 sandwiches the multilayer body with the lower wall 312 (the base member).

The receiving case 33 is a box body. The receiving case 33 receives the humidification chamber 40 from the upper side. The receiving case 33 includes an opening 3320. The thermal conductor 60 is upwardly exposed from the opening 3320. With this, the humidification chamber 40 received within the receiving case 33 is in contact with the thermal conductor 60. Accordingly, the CPAP apparatus 10 can heat the liquid stored within the humidification chamber 40 and attain a gas flow with the desired humidity.

### (Base Member)

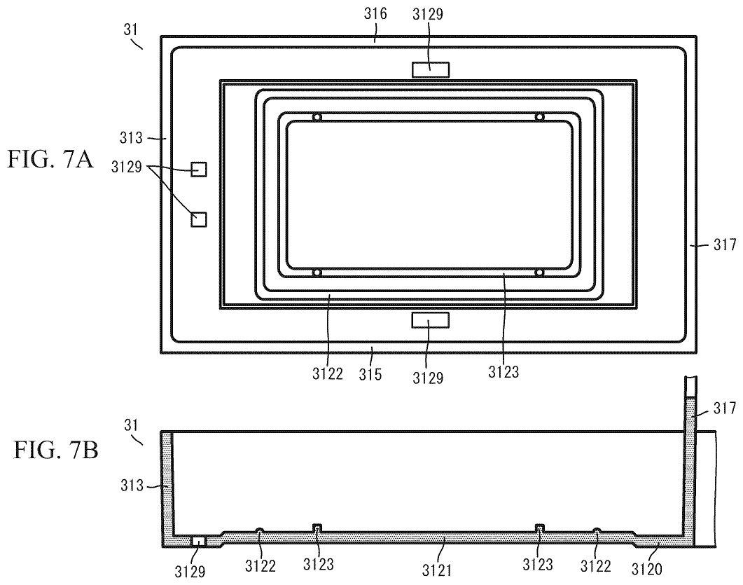

FIG. 7A is a plan view of the box body including the base member according to the embodiment of the present disclosure. FIG. 7B is a side sectional view of this box body.

The lower wall 312 is made of a material (for example, a resin) having sufficient hardness to resist deformation by external force. The lower wall 312 corresponds to the base member in the present disclosure. Note that the base member may be the lower wall 312 itself or may be a member which is different from the lower wall 312 and which is fixed to the lower wall 312. The lower wall 312 includes an outer peripheral wall 3120, a center wall 3121, a protruding portion 3122, and a protruding portion 3123. The outer peripheral wall 3120 has a frame shape in plan view. The center wall 3121 has a rectangular shape in plan view. The center wall 3121 is connected to an inner peripheral end of the outer peripheral wall 3120. The center wall 3121 is disposed on an upper side with respect to the outer peripheral wall 3120. In other words, the outer peripheral wall 3120 has a shape downwardly recessed with respect to the center wall 3121 in a vertical direction.

The protruding portion 3122 is formed on the center wall 3121. The protruding portion 3122 protrudes from an upper surface of the center wall 3121. The protruding portion 3122 has a frame shape. The protruding portion 3122 corresponds to a fifth protruding portion in the present disclosure.

The protruding portion 3123 is formed on the center wall 3121. The protruding portion 3123 protrudes from the upper surface of the center wall 3121. The protruding portion 3123 has a frame shape. The protruding portion 3123 is formed inside the protruding portion 3122 when the lower wall 312 is viewed in plan view.

A plurality of discharge ports 3129 are formed in the outer peripheral wall 3120. The discharge ports 3129 extend through the wall.

### (Receiving Case)

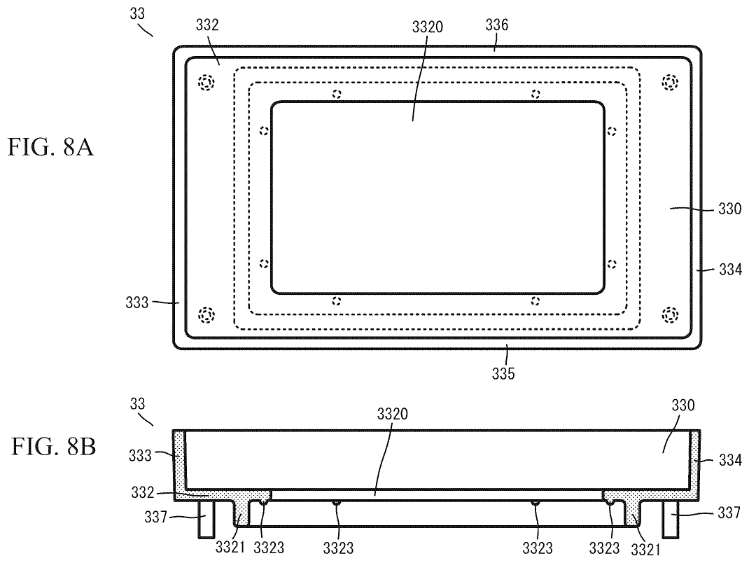

FIG. 8A is a plan view of the receiving case according to the embodiment of the present disclosure. FIG. 8B is a side sectional view of the receiving case.

The receiving case 33 is made of a material (for example, a resin) having sufficient hardness to resist deformation by external force. The receiving case 33 includes a lower wall 332, a side wall 333, a side wall 334, a side wall 335, and a side wall 336. The side wall 333, the side wall 334, the side wall 335, and the side wall 336 extend upwardly from the lower wall 332. The side wall 333, the side wall 334, the side wall 335, and the side wall 336 are connected to four sides of the lower wall 332. With this, the receiving case 33 includes the receiving space 330 receiving the humidification chamber 40.

The lower wall 332 includes an opening 3320 positioned at a center when the receiving case is viewed in plan view. The opening 3320 extends through the lower wall 332 in the thickness direction.

A protruding portion 3321 is formed on the lower wall 332. The protruding portion 3321 protrudes from the lower surface of the lower wall 332. The protruding portion 3321 has a frame shape.

A plurality of protruding portions 3323 are formed on the lower wall 332. Each of the plurality of protruding portions 3323 has, for example, a hemisphere shape. The plurality of protruding portions 3323 are disposed in a frame shape. In other words, the protruding portions 3323 are partially formed in part of the frame shape. The plurality of protruding portions 3323 are formed inside the protruding portion 3321 when the lower wall 332 is viewed in plan view. The frame shape in which the plurality of protruding portions 3323 are disposed is substantially identical to the frame shape formed by the protruding portions 3122 of the base member. The protruding portions 3323 corresponds to a first protruding portion in the present disclosure.

The lower wall 332 is provided with a plurality of foot portions 337. The plurality of foot portions 337 have a shape extending downwardly from the lower wall 332. The plurality of foot portions 337 are connected to the lower wall 332.

### (Thermal Conductor)

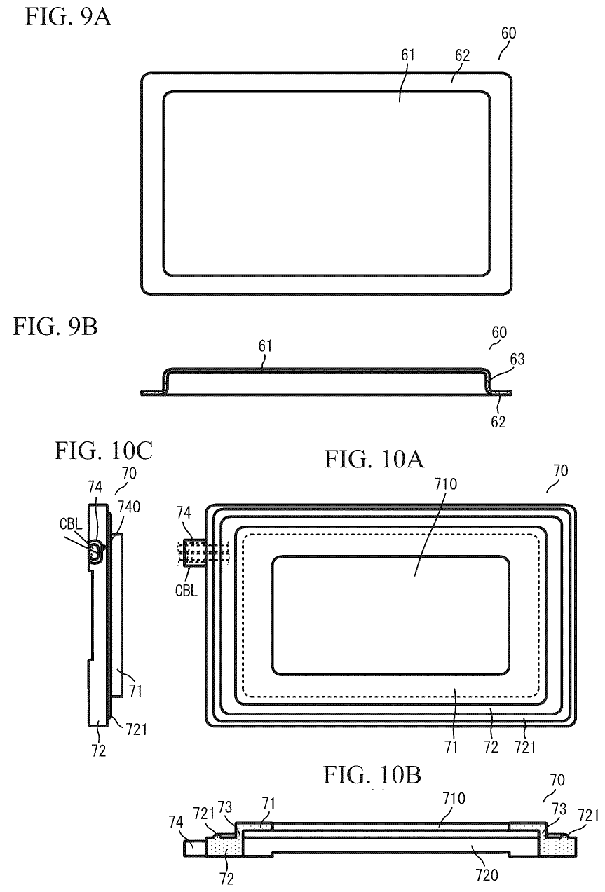

FIG. 9A is a plan view of a thermal conductor according to the embodiment of the present disclosure. FIG. 9B is a side sectional view of the thermal conductor.

The thermal conductor 60 is made of a material (for example, a metal) having high thermal conductivity. The thermal conductor 60 includes a main flat plate 61, a peripheral portion 62, and a side wall 63.

The main flat plate 61 has a rectangular shape in plan view. The peripheral portion 62 has a frame shape extending along an outer periphery of the main flat plate 61. The peripheral portion 62 is disposed on the lower side with respect to the main flat plate 61. An outer peripheral end of the main flat plate 61 and an inner peripheral end of the peripheral portion 62 are positioned at a substantially identical position in plan view. The side wall 63 connects the outer peripheral end of the main flat plate 61 and the inner peripheral end of the peripheral portion 62.

### (Elastic Member 70)

FIG. 10A is a plan view of the elastic member according to the embodiment of the present disclosure. FIG. 10B is a first side sectional view of the elastic member. FIG. 10C is a second side view of the elastic member.

The elastic member 70 is made of a material (for example, a flexible material such as an elastic material) which is more deformable by external force than the base member and the receiving case 33. The elastic member 70 includes a first main body 71, a second main body 72, and a connection portion 73.

The first main body 71 is a flat plate. The first main body 71 includes an opening 710 positioned at a center in plan view. The opening 710 extends through the first main body 71 in the thickness direction.

The second main body 72 has a frame shape extending along an outer periphery of the first main body 71. The second main body 72 includes an opening 720. The second main body 72 is disposed on the lower side with respect to the main flat plate 71. An outer peripheral end of the first main body 71 and an inner peripheral end of the second main body 72 are positioned at a substantially identical position in plan view. The connection portion 73 connects the outer peripheral end of the first main body 71 and the inner peripheral end of the second main body 72.

A protruding portion 721 is formed on the second main body 72. The protruding portion 721 protrudes from an upper surface of the second main body 72. The protruding portion 721 has a frame shape. The frame shape formed by the protruding portion 721 is substantially identical to the frame shape in which the plurality of protruding portions 3323 of the receiving case 33 are disposed and the frame shape formed by the protruding portions 3122 of the base member. The protruding portion 721 corresponds to a third protruding portion in the present disclosure.

### (More Specific Configuration of Receiving Unit)

As illustrated in FIG. 5 and FIG. 6, the elastic member 70 is disposed on the upper side of the lower wall 312 which is the base member. In this case, a lower surface of the second main body 72 of the elastic member 70 is in contact with the protruding portion 3122 of the lower wall 312. With this, the elastic member 70 is supported on the upper side of the lower wall 312.

The circuit board 500 is disposed on the lower surface of the first main body 71 of the elastic member 70. In other words, the upper surface of the circuit board 500 is abutted on the first main body 71. The heating element 50 is electrically and physically mounted on the lower surface of the circuit board 500. The heating element 50 may be mounted on the upper surface of the circuit board 500.

The thermal conductor 60 covers the heating element 50, the circuit board 500, and the elastic member 70 from the upper side. More specifically, the main flat plate 61 of the thermal conductor 60 overlaps the heating element 50 and the circuit board 500 when the receiving unit is viewed in plan view. The side wall 63 of the thermal conductor 60 covers the side of the heating element 50 and the side of the circuit board 500. In this case, the side wall 63 may cover a portion near the upper end of the first main body 71 of the elastic member 70. The peripheral portion 62 of the thermal conductor 60 covers the second main body 72 of the elastic member 70 from the upper side.

The lower surface of the main flat plate 61 of the thermal conductor 60 is in contact with the heating element 50. The main flat plate 61 and the heating element 50 need not be in direct contact with each other as long as the main flat plate 61 and the heating element 50 are thermally in contact with each other. However, it is preferable that the thermal loss due to this contact portion be low.

The lower surface of the peripheral portion 62 of the thermal conductor 60 is in contact with the protruding portion 721 of the second main body 72 of the elastic member 70.

The receiving case 33 is disposed on the upper side of the lower wall 312 which is the base member. The receiving case 33 is fixed to the lower wall 312 by the plurality of foot portions 337.

In this case, the main flat plate 61 of the thermal conductor 60 is upwardly exposed from the opening 3320 of the lower wall 332 of the receiving case 33.

Moreover, the plurality of protruding portions 3323 of the receiving case 33 is in contact with the upper surface of the peripheral portion 62 of the thermal conductor 60. With this configuration, the peripheral portion 62 of the thermal conductor 60 is sandwiched and fixed by the plurality of protruding portions 3323 of the receiving case 33 and the protruding portion 721 of the second main body 72 of the elastic member 70.

Furthermore, the second main body 72 of the elastic member 70 is sandwiched and fixed by the peripheral portion 62 of the thermal conductor 60 and the protruding portion 3122 of the lower wall 312.

### (Discharge of Water)

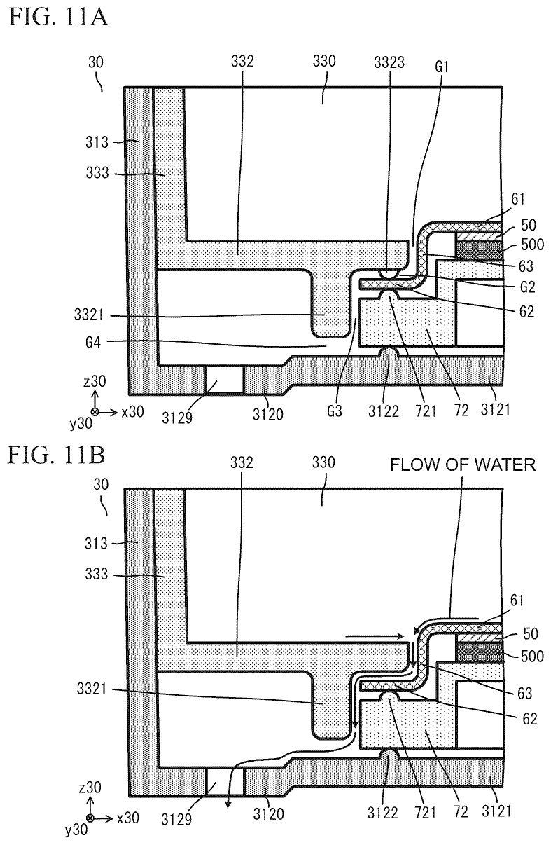

FIG. 11A and FIG. 11B are each an enlarged view of a side cross section for explaining a water discharge mechanism of the CPAP apparatus according to the embodiment of the present disclosure. FIG. 11A is an illustration for readily understanding a gap (flow path) configured to discharge water. FIG. 11B is an illustration for readily understanding one example of a flow of water.

An outer shape of the main flat plate 61 of the thermal conductor 60 is smaller than the opening 3320 of the receiving case 33 (see FIG. 5 and FIG. 6). Accordingly, as illustrated in FIG. 11A, a gap G1 is formed between an outer surface of the side wall 63 of the thermal conductor 60 and an inner side surface of the lower wall 332 of the receiving case 33 on the opening 3320 side.

The lower wall 332 of the receiving case 33 is in contact with the peripheral portion 62 of the thermal conductor 60 with the plurality of protruding portions 3323 therebetween. Accordingly, as illustrated in FIG. 11A, a gap G2 is formed between the upper surface of the peripheral portion 62 of the thermal conductor 60 and the lower surface of the lower wall 332 of the receiving case 33.

When the CPAP apparatus 10 is viewed in plan view, a size of the frame shape of the inner peripheral surface of the protruding portion 3321 of the lower wall 332 of the receiving case 33 is greater than a size of the frame shape of the outer peripheral surface of the peripheral portion 62 of the thermal conductor 60 and a size of the frame shape of the outer peripheral surface of the second main body 72 of the elastic member 70. Accordingly, as illustrated in FIG. 11A, a gap G3 is formed between the inner peripheral surface of the protruding portion 3321 of the lower wall 332 of the receiving case 33 and each of the outer peripheral surface of the peripheral portion 62 of the thermal conductor 60 and the outer peripheral surface of the second main body 72 of the elastic member 70.

The protruding portion 3321 of the lower wall 332 of the receiving case 33 is not abutted on the upper surface of the lower wall 312 (the center wall 3121) which is the base member. Accordingly, as illustrated in FIG. 11A, a gap G4 is formed between the protruding portion 3321 of the lower wall 332 of the receiving case 33 and the lower wall 312 (the center wall 3121) which is the base member.

In this case, when water enters (spills into) the receiving space 330, the water flows onto the outer peripheral wall 3120 of the lower wall 312 through a space defined by the gap G1, a space defined by the gap G2 (portions in which the plurality of protruding portions 3323 are not formed), a space defined by the gap G3, and a space defined by the gap G4. The discharge ports 3129 are formed in the outer peripheral wall 3120. Accordingly, the water flowing onto the outer peripheral wall 3120 is discharged to the outside of the CPAP apparatus 10.

In this way, the CPAP apparatus 10 can discharge the water entering the receiving space 330 in which the humidification chamber 40 is disposed to the outside.

In this case, for example, as illustrated in FIG. 11B, the heating element 50 and the circuit board 500 are isolated from the flow path of water by the thermal conductor 60 and the elastic member 70. Consequently, the CPAP apparatus 10 can ensure the watertightness for the heating element 50 and the circuit board 500.

Moreover, the frame-shaped protruding portion 721 of the elastic member 70 is pressed and deformed by a predetermined amount by the thermal conductor 60. Therefore, it is possible to improve the close contact between the thermal conductor 60 and the protruding portion 721 of the elastic member 70. Furthermore, the CPAP apparatus 10 can ensure watertightness even when the shapes of the members that constitute the CPAP apparatus 10, such as the receiving case 33, the thermal conductor 60, and the lower wall 312 which is the base member, are deformed. Consequently, the CPAP apparatus 10 can ensure higher watertightness for the heating element 50 and the circuit board 500.

Moreover, the lower surface of the elastic member 70 is supported by the frame-shaped protruding portion 3122 of the lower wall 312. With this, the CPAP apparatus 10 can suppress water from reaching the heating element 50 and the circuit board 500 through a portion between the elastic member 70 and the lower wall 312 and the internal space of the elastic member 70. Consequently, the CPAP apparatus 10 can further improve the watertightness for the heating element 50 and the circuit board 500.

Furthermore, in this configuration, it is possible to ensure the discharge of water to the outside of the CPAP apparatus 10 and the watertightness for the heating element 50 and the circuit board 500 only by providing the protruding portions on the plurality of constituting elements relating to the heating mechanism and providing the openings each having an appropriate size. With this, the CPAP apparatus 10 can ensure the discharge of water to the outside of the CPAP apparatus 10 and the watertightness for the heating element 50 and the circuit board 500 by the simple configuration.

Moreover, in this configuration, the heating element 50 is in contact with the thermal conductor 60. The heating element 50 is not in contact with the lower wall 312 or the receiving case 33. Therefore, the heat from the heating element 50 is efficiently transmitted to the thermal conductor 60. The CPAP apparatus 10 can efficiently heat the humidification chamber 40 and improve the humidification efficiency. In this case, when the thermal conductivity of the elastic member 70 is low, less heat from the heating element 50 leaks to the lower side. Accordingly, the CPAP apparatus 10 can more efficiently heat the humidification chamber 40 and further improve the humidification efficiency.

Furthermore, as illustrated in FIG. 10A, FIG. 10B, and FIG. 10C, the elastic member 70 is provided with a wiring cover 74. The wiring cover 74 includes a through hole 740. The through hole 740 extends between an outer side and an inner side of the second main body 72 of the elastic member 70 in the lateral direction. An opening sectional area of the through hole 740 (an area perpendicular to a direction in which the through hole 740 extends) is smaller than a sectional area of a cable CBL inserted through the through hole 740. In a case in which there are a plurality of cables CBL inserted through the through hole 740, the opening sectional area is smaller than a sum of areas of the cables CBL inserted through the through hole 740. With this, when the cable CBL is inserted through the through hole 740, the inner wall surface of the through hole 740 is in close contact with a peripheral surface of the cable CBL. Accordingly, it is possible to ensure the watertightness for the circuit board 500 and the heating element 50 while the CPAP apparatus 10 has a structure in which the cable CBL is inserted from the outer side of the elastic member 70 to the inner side of the elastic member 70 and is connected to the circuit board 500.

### (Variations of Protruding Portions)

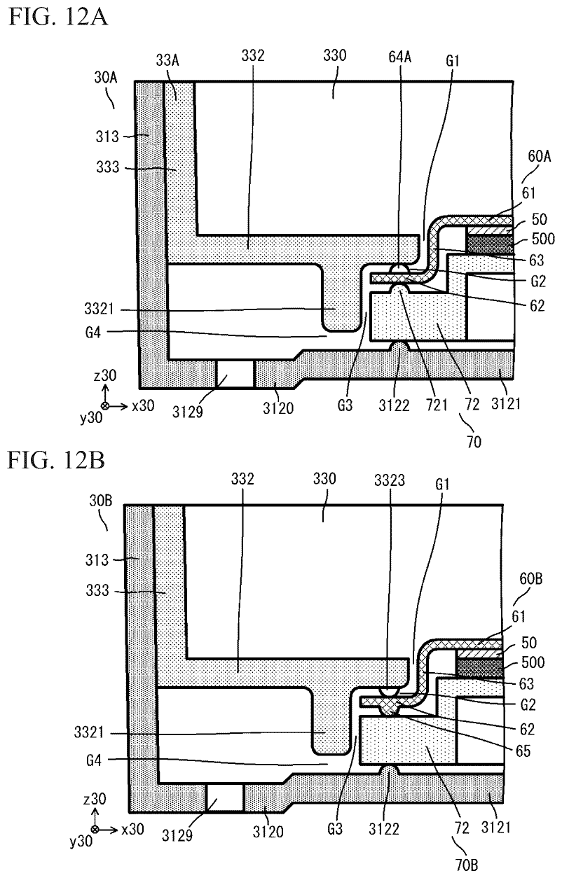

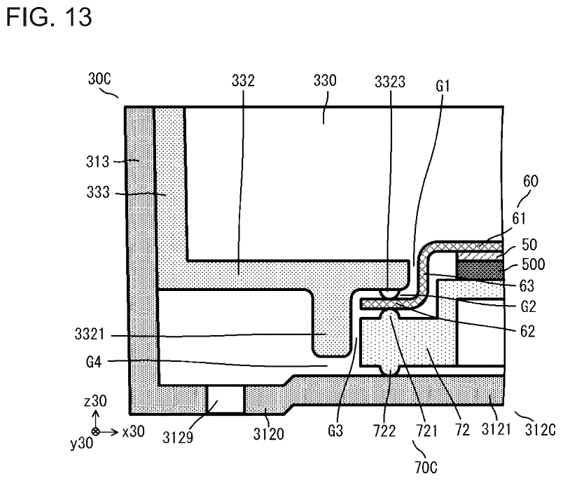

FIG. 12A, FIG. 12B, and FIG. 13 are each an enlarged view of a side cross section illustrating a variation of the protruding portions of the CPAP apparatus according to the embodiment of the present disclosure.

In a base unit 30A of FIG. 12A, a plurality of protruding portions 64A between a receiving case 33A and a peripheral portion 62 of a thermal conductor 60A is formed on an upper surface of the peripheral portion 62 of the thermal conductor 60A. The protruding portions 64A correspond to second protruding portions in the present disclosure.

In a base unit 30B of FIG. 12B, a protruding portion 65 between an elastic member 70B and a peripheral portion 62 of a thermal conductor 60B is formed on a lower surface of the peripheral portion 62 of the thermal conductor 60B. The protruding portion 65 corresponds to a fourth protruding portion in the present disclosure.

In a base unit 30C of FIG. 13, a protruding portion 722 between an elastic member 70C and a center wall 3121 of a lower wall 312C is formed on a lower surface of the elastic member 70C. The protruding portion 722 corresponds to a sixth protruding portion in the present disclosure.

These configurations can also attain operations and effects similar to those of the configuration of the above-described CPAP apparatus 10.

It is possible to appropriately combine these configurations.

(1) A CPAP apparatus includes: a housing; a fan mechanism provided in the housing and configured to generate a gas flow to be supplied to a user; a humidification chamber detachably provided in the housing and configured to store water for humidifying the gas flow; and a receiving unit which is provided in the housing and which receives the humidification chamber, the receiving unit including a base member formed by a portion of the housing or fixed to the housing, a receiving case which is disposed on an upper side of the base member and which includes an opening formed in a lower wall, a heating unit disposed between the base member and the lower wall of the receiving case, and an elastic member which is disposed on the base member and which holds the heating unit, the heating unit including a heating element, and a thermal conductor which is disposed on an upper side of the heating element, which has a shape wider than a shape of the heating element when the heating element is viewed from the receiving unit in plan view, and which includes a peripheral portion that does not overlap the heating element, the lower wall of the receiving case including a plurality of first protruding portions formed on a portion overlapping an outer peripheral portion of the thermal conductor in plan view and arranged at intervals in a peripheral direction of the outer peripheral portion, the lower wall of the receiving case and the thermal conductor being in contact with each other with the plurality of first protruding portions therebetween.

(2) A CPAP apparatus includes: a housing; a fan mechanism provided in the housing and configured to generate a gas flow to be supplied to a user; a humidification chamber detachably provided in the housing and configured to store water for humidifying the gas flow; and a receiving unit which is provided in the housing and which receives the humidification chamber; the receiving unit including a base member formed by a portion of the housing or fixed to the housing, a receiving case which is disposed on an upper side of the base member and which includes an opening formed in a lower wall, a heating unit disposed between the base member and the lower wall of the receiving case, and an elastic member which is disposed on the base member and which holds the heating unit, the heating unit including a heating element, and a thermal conductor which is disposed on an upper side of the heating element, which has a shape wider than a shape of the heating element when the heating element is viewed from the receiving unit in plan view, and which includes a peripheral portion that does not overlap the heating element, the thermal conductor including a plurality of second protruding portions formed on a portion overlapping an outer peripheral portion of the thermal conductor in plan view and arranged at intervals in a peripheral direction of the outer peripheral portion, the lower wall of the receiving case and the thermal conductor being in contact with each other with the plurality of second protruding portions therebetween.

(3) The CPAP apparatus according to (1) or (2), wherein the elastic member includes a third protruding portion which has a frame shape to surround the heating element and which overlaps the outer peripheral portion when the heating element is viewed from the receiving unit in plan view, and the thermal conductor and the elastic member are in contact with each other with the third protruding portion therebetween.

(4) The CPAP apparatus according to any one of (1) to (3), wherein the thermal conductor includes a fourth protruding portion which has a frame shape to surround the heating element and which overlaps the outer peripheral portion when the heating element is viewed from the receiving unit in plan view, and the thermal conductor and the elastic member are in contact with each other with the fourth protruding portion therebetween.

(5) The CPAP apparatus according to any one of (1) to (4), wherein the base member includes a fifth protruding portion which has a frame shape to surround the heating element and which overlaps the outer peripheral portion when the heating element is viewed from the receiving unit in plan view, and the base member and the elastic member are in contact with each other with the fifth protruding portion therebetween.

(6) The CPAP apparatus according to any one of (1) to (5), wherein the elastic member includes a sixth protruding portion which has a frame shape to surround the heating element and which overlaps the outer peripheral portion when the heating element is viewed from the receiving unit in plan view, and the base member and the elastic member are in contact with each other with the sixth protruding portion therebetween.

(7) The CPAP apparatus according to any one of (1) to (6), wherein the base member includes a center wall overlapping the heating unit and the elastic member when the CPAP apparatus is viewed in plan view, and an outer peripheral wall disposed around the center wall and positioned on a lower side with respect to the center wall, and the outer peripheral wall includes a discharge port.

(8) The CPAP apparatus according to any one of (1) to (7), wherein the CPAP apparatus includes a cable connected to the heating element, the elastic member includes a through hole extending through a side wall in a lateral direction, and the through hole is in close contact with the cable in a state in which the cable is inserted through the through hole.
10: CPAP apparatus
20: main unit
30, 30A, 30B, 30C: base unit
31: box body
32: lid
33: receiving case
33A: receiving case
35: gas blowing pipe
36: inlet port
38: exhaust port
39: inlet port
40: humidification chamber
50: heating element
60, 60A, 60B: thermal conductor
61: main flat plate
62: peripheral portion
63: side wall
64A: protruding portion
65: protruding portion
70, 70B, 70C: elastic member
71: first main body
72: second main body
73: connection portion
74: wiring cover
281: inlet port
291: exhaust port
311: upper wall
312, 312C: lower wall
313, 314, 315, 316, 317: side wall
321: upper wall
323, 325, 326: side wall
330: receiving space
332: lower wall
333, 334, 335, 336: side wall
337: foot portion
350: opening
360: hook
361: operator
500: circuit board
710, 720: opening
721, 722: protruding portion
740: through hole
3120: outer peripheral wall
3121: center wall
3122, 3123, 3321, 3323: protruding portion
3129: discharge port
3170: receiving member
3320: opening
CBL: cable
G1, G2, G3, G4: gap
S31A, S31B, S32: space

1. A CPAP apparatus comprising:
a housing;
a fan mechanism provided in the housing and configured to generate a gas flow to be supplied to a user;
a humidification chamber detachably provided in the housing and configured to store water for humidifying the gas flow; and
a receiving unit provided in the housing and receiving the humidification chamber,
the receiving unit including
a base member provided by a portion of the housing or fixed to the housing,
a receiving case disposed on an upper side of the base member and including an opening provided in a lower wall,
a heating unit disposed between the base member and the lower wall of the receiving case, and
an elastic member disposed on the base member and holding the heating unit,
the heating unit including
a heating element, and
a thermal conductor disposed on an upper side of the heating element, having a shape wider than a shape of the heating element when the heating element is viewed from the receiving unit in plan view, and including a peripheral portion not overlapping the heating element,
the lower wall of the receiving case including a plurality of first protruding portions provided on a portion overlapping an outer peripheral portion of the thermal conductor in plan view, the plurality of first protruding portions being arranged at intervals in a peripheral direction of the outer peripheral portion,
the lower wall of the receiving case and the thermal conductor being in contact with each other with the plurality of first protruding portions therebetween.

2. A CPAP apparatus comprising:
a housing;
a fan mechanism provided in the housing and configured to generate a gas flow to be supplied to a user;
a humidification chamber detachably provided in the housing and configured to store water for humidifying the gas flow; and
a receiving unit provided in the housing and receiving the humidification chamber;
the receiving unit including
a base member provided by a portion of the housing or fixed to the housing,
a receiving case disposed on an upper side of the base member and including an opening provided in a lower wall,
a heating unit disposed between the base member and the lower wall of the receiving case, and
an elastic member disposed on the base member and holding the heating unit,
the heating unit including
a heating element, and
a thermal conductor disposed on an upper side of the heating element, having a shape wider than a shape of the heating element when the heating element is viewed from the receiving unit in plan view, and including a peripheral portion not overlapping the heating element,
the thermal conductor including a plurality of second protruding portions provided on an outer peripheral portion of the thermal conductor in plan view, the plurality of second protruding portions being arranged at intervals in a peripheral direction of the outer peripheral portion,
the lower wall of the receiving case and the thermal conductor being in contact with each other with the plurality of second protruding portions therebetween.

3. The CPAP apparatus according to claim 1, wherein
the elastic member includes a third protruding portion having a frame shape to surround the heating element and overlapping the outer peripheral portion when the heating element is viewed from the receiving unit in plan view, and
the thermal conductor and the elastic member are in contact with each other with the third protruding portion therebetween.

4. The CPAP apparatus according to claim 1, wherein
the thermal conductor includes a fourth protruding portion provided on the outer peripheral portion of the thermal conductor in plan view and the fourth protruding portion has a frame shape to surround the heating element, and
the thermal conductor and the elastic member are in contact with each other with the fourth protruding portion therebetween.

5. The CPAP apparatus according to claim 1, wherein
the base member includes a fifth protruding portion which has a frame shape to surround the heating element and which overlaps the outer peripheral portion when the heating element is viewed from the receiving unit in plan view, and
the base member and the elastic member are in contact with each other with the fifth protruding portion therebetween.

6. The CPAP apparatus according to claim 1, wherein
the elastic member includes a sixth protruding portion having a frame shape to surround the heating element and overlapping the outer peripheral portion when the heating element is viewed from the receiving unit in plan view, and
the base member and the elastic member are in contact with each other with the sixth protruding portion therebetween.

7. The CPAP apparatus according to claim 1, wherein
the base member includes
a center wall overlapping the heating unit and the elastic member when the CPAP apparatus is viewed in plan view, and
an outer peripheral wall disposed around the center wall and positioned on a lower side with respect to the center wall, and
the outer peripheral wall includes a discharge port.

8. The CPAP apparatus according to claim 1, wherein
the CPAP apparatus includes a cable connected to the heating element,
the elastic member includes a through hole extending through a side wall in a lateral direction, and
the through hole is in close contact with the cable in a state in which the cable is inserted through the through hole.

9. The CPAP apparatus according to claim 2, wherein
the elastic member includes a third protruding portion having a frame shape to surround the heating element and overlapping the outer peripheral portion when the heating element is viewed from the receiving unit in plan view, and
the thermal conductor and the elastic member are in contact with each other with the third protruding portion therebetween.

10. The CPAP apparatus according to claim 2, wherein
the thermal conductor includes a fourth protruding portion provided on the outer peripheral portion of the thermal conductor in plan view and the fourth protruding portion has a frame shape to surround the heating element, and
the thermal conductor and the elastic member are in contact with each other with the fourth protruding portion therebetween.

11. The CPAP apparatus according to claim 3, wherein
the thermal conductor includes a fourth protruding portion provided on the outer peripheral portion of the thermal conductor in plan view and the fourth protruding portion has a frame shape to surround the heating element, and
the thermal conductor and the elastic member are in contact with each other with the fourth protruding portion therebetween.

12. The CPAP apparatus according to claim 2, wherein
the base member includes a fifth protruding portion which has a frame shape to surround the heating element and which overlaps the outer peripheral portion when the heating element is viewed from the receiving unit in plan view, and
the base member and the elastic member are in contact with each other with the fifth protruding portion therebetween.

13. The CPAP apparatus according to claim 3, wherein
the base member includes a fifth protruding portion which has a frame shape to surround the heating element and which overlaps the outer peripheral portion when the heating element is viewed from the receiving unit in plan view, and
the base member and the elastic member are in contact with each other with the fifth protruding portion therebetween.

14. The CPAP apparatus according to claim 4, wherein
the base member includes a fifth protruding portion which has a frame shape to surround the heating element and which overlaps the outer peripheral portion when the heating element is viewed from the receiving unit in plan view, and
the base member and the elastic member are in contact with each other with the fifth protruding portion therebetween.

15. The CPAP apparatus according to claim 2, wherein
the elastic member includes a sixth protruding portion having a frame shape to surround the heating element and overlapping the outer peripheral portion when the heating element is viewed from the receiving unit in plan view, and
the base member and the elastic member are in contact with each other with the sixth protruding portion therebetween.

16. The CPAP apparatus according to claim 3, wherein
the elastic member includes a sixth protruding portion having a frame shape to surround the heating element and overlapping the outer peripheral portion when the heating element is viewed from the receiving unit in plan view, and
the base member and the elastic member are in contact with each other with the sixth protruding portion therebetween.

17. The CPAP apparatus according to claim 4, wherein
the elastic member includes a sixth protruding portion having a frame shape to surround the heating element and overlapping the outer peripheral portion when the heating element is viewed from the receiving unit in plan view, and
the base member and the elastic member are in contact with each other with the sixth protruding portion therebetween.

18. The CPAP apparatus according to claim 5, wherein
the elastic member includes a sixth protruding portion having a frame shape to surround the heating element and overlapping the outer peripheral portion when the heating element is viewed from the receiving unit in plan view, and
the base member and the elastic member are in contact with each other with the sixth protruding portion therebetween.

19. The CPAP apparatus according to claim 2, wherein
the base member includes
a center wall overlapping the heating unit and the elastic member when the CPAP apparatus is viewed in plan view, and
an outer peripheral wall disposed around the center wall and positioned on a lower side with respect to the center wall, and
the outer peripheral wall includes a discharge port.

20. The CPAP apparatus according to claim 3, wherein
the base member includes
a center wall overlapping the heating unit and the elastic member when the CPAP apparatus is viewed in plan view, and
an outer peripheral wall disposed around the center wall and positioned on a lower side with respect to the center wall, and
the outer peripheral wall includes a discharge port.
